(19) United States
(12) Patent Application Publication
(10) Pub. No.: US 2013/0165368 A1
(43) Pub. Date: Jun. 27, 2013

(54) ANTIMICROBIAL COMPOUNDS

(71) Applicants: Ian Steel, Leeds (GB); Paul Armstrong, Newcastle (GB)

(72) Inventors: Ian Steel, Leeds (GB); Paul Armstrong, Newcastle (GB)

(21) Appl. No.: 13/576,199

(22) PCT Filed: Feb. 1, 2011
(86) PCT No.: PCT/GB11/00129

§ 371 Date: Oct. 12, 2012

(30) Foreign Application Priority Data
Feb. 2, 2010 (GB) 1001632.7

(51) Int. Cl. A61K 31/08; A61K 31/496; A61K 31/505; A61K 31/5383; A61K 31/575; A61K 31/351; A61K 31/4025; A61K 31/546; A61K 31/7036; A61K 31/165; A61K 31/65; A61K 31/7048; A61K 45/06; A61K 31/133; A61K 38/14

(52) U.S. Cl. CPC A61K 31/08; A61K 38/14; A61K 31/496; A61K 31/505; A61K 31/5383; A61K 31/575; A61K 31/351; A61K 31/4025; A61K 31/546; A61K 31/7036; A61K 31/165; A61K 31/65; A61K 31/7048; A61K 45/06; A61K 31/133
USPC 514/2.3; 568/678; 514/723; 514/254.11; 514/275; 514/230.2; 514/171; 514/460; 514/422; 514/201; 514/40; 514/628; 514/154; 514/29; 564/508; 514/670; 568/679

(57) ABSTRACT

A composition for use in the prevention and/or treatment of microbial infection comprising essentially of one or more pure alkanol alkoxylates, diol alkoxylates and/or triol alkoxylates.

The present invention relates to the prevention and treatment of infection, especially bacterial infection and/or superinfection, by use of antimicrobial compounds.

An infection is defined to be a pathological state or a manifestation of diseases in a certain part of the body, and is due to external invasion of pathogenic or disease-causing micro-organisms. Human and animal bodies (as the host organism) will respond adversely to such an invasion when such foreign organisms colonize and attack their bodies, leading to a state of infection. The invading organisms may be in the form of virus, bacteria or yeast.

A common bacterium found on humans is Staphylococcus aureus, which is typically found on external skin surfaces and in the upper respiratory tract, particularly the nasal passages. Healthy individuals are usually unaware of staphylococcal carriage but they may suffer from minor skin infections such as boils and abscesses. However because Staphylococcus aureus is an opportunist pathogen, given the right circumstances it can cause more serious infections. In particular burns and surgical wound infections are commonly invaded by Staphylococcus aureus, where the production of toxins by this bacteria can, for example, give rise to toxic shock syndrome leading to fever, sickness and in some cases death.

In the case of bacterial infection, such as infection caused by Staphylococcus aureus, before the 1950's known treatments involved the administration of benzylpenicillin, but by the late 1950's certain strains of Staphylococcus aureus were resistant to benzylpenicillin. This resistance resulted in the synthesis of methicillin in which the phenol group of benzylpenicillin is disubstituted with methoxy groups. Unfortunately, in what has been observed as an increasing trend, as soon as methicillin was used clinically, methicillin-resistant Staphylococcus aureus (MRSA) strains were isolated resulting in a further line of bacterial resistance.

Indeed the use of different types of antibiotics over the years has lead to the emergence of multi-resistant MRSA strains and in some cases, currently the only option for antimicrobial therapy is to use the glycopeptide antibiotic vancomycin. However it has now been observed that Staphylococcus aureus also exhibits vancomycin-resistance. As a result the search for new anti-staphylococcal agents, and antimicrobial agents more generally, is a pressing issue.

In the past, there has been research into the effect of polidocanol to suppress the resistance to methicillin and oxacillin in Staphylococcus aureus. In a paper entitled “Suppression of Intrinsic Resistance to Penicillins in Staphylococcus Aureus by Polidocanol, a Dodecyl Polyethyleneoxid Ether” by Bruns et al. in the journal Antimicrobial Agents and Chemotherapy, April 1985, Vol. 27, No. 4, pages 632-639, it is reported that polidocanol, which the authors define as being Laureth-9, exerts no antimicrobial inhibitory effects.

Conversely in a paper entitled “The Intrinsic Antimicrobial Activity of Selected Sclerosing Agents in Sclerotherapy” in the 1996 Journal by the American Society for Dermatologic Surgery, Inc. pages 369-371, Vol. 22, by Sadick et al., it is stated that polidocanol exerts activity against two strains of Staphylococcus aureus, however this paper is not clear as to which strains of Staphylococcus aureus have been studied. Furthermore there is no definition in Sadick et al. of exactly what is meant by “polidocanol” (i.e. exactly which compounds were used to conduct the experiments) which is important for the reasons to be highlighted below.

Polidocanol is a commercially available product. However it is not a straightforward chemical of uniform composition, but instead it is a mixture of different ethoxylated alcohols of differing length having differing numbers of ethoxy-groups. Thus the name “polidocanol” does not refer to a specific compound or well defined mixture. Consequently the reference to polidocanol being Laureth-9, i.e. C₁₂H₂₅(—O—C₂H₄—)₉OH (which may be described as ethoxylated lauryl alcohol having nine ethoxy groups—nonaethylene glycol monododecyl ether) by Bruns et al., and the omission in Sadick et al. to even define what is meant by polidocanol, are both misleading at best. For instance, the literature states that polidocanol is not a simple, single, discrete compound (i.e. Laureth-9) but instead is a complex variable multi-component mixture of ethoxylates of lauryl alcohol and lauryl alcohol (i.e. dodecyl alcohol, C₁₂H₂₆O) itself. A further complication lies in the fact that commercially available laureth products will almost certainly have been synthesised from a 12-carbon chain (lauryl) alcohol feedstock that was not 100% w/w pure—the feedstock would most likely contain other alcohols of differing carbon chain length (e.g. C₁₀ and C₁₄) which will also be ethoxylated to varying degrees.

Determination of the actual composition of polidocanol is further complicated if reference is made to its CAS number: 3055-99-0. Compare this to the CAS number of Laureth-9 (which according to Bruns et al. is the same as polidocanol) of 9066-65-9 and it becomes clear that there are glaring inconsistencies in the way that polidocanol and indeed Laureth-9 have been not only cited in the prior art, but presumably also experimented with.

Moreover, as described above, it has been observed that polidocanol includes a significant proportion of lauryl alcohol, which, importantly, is known to exert potent antimicrobial properties even at low concentrations. Thus to a person skilled in the art, it is unclear as to (i) the exact formulation and composition of polidocanol that has been used in experiments to date and (ii) regardless of the composition of the ethoxylated component, whether it is indeed the lauryl alcohol itself that is proving successful in its antimicrobial effect.

We are therefore faced with two major difficulties when studying the effects of polidocanol and its antimicrobial properties. Firstly, all commonly commercially available materials are complex and variable mixtures, thus simply referring to polidocanol does not provide a sufficient indication of exactly what species of ethoxylated lauryl alcohols are contained within the species being studied. Secondly, the commercially available mixtures appear to contain significant amounts of free lauryl alcohol. It would therefore be desirable to provide a composition that is readily identifiable and successful in the treatment and prevention of infection and super-infection and which is free of the uncertainty that has been exhibited in the prior art.

Accordingly the present invention provides a composition for use in the treatment and/or prevention of microbial infection comprising essentially of one or more pure alkanol alkoxylates, diol alkoxylates and/or triol alkoxylates.

By “comprising essentially of one or more pure alkanol alkoxylates, diol alkoxylates and/or triol alkoxylates” and like terms, it is meant that the biologically active agent of the composition is a pure alkanol alkoxylate, diol alkoxylate and/or triol alkoxylate, having a GC purity (i.e. the purity as determined by Gas Chromatography) of at least 45%. The remaining 55% or less (as also determined by gas chromatography) of the biologically active agent of the composition may be composed of other species, including the basic (i.e. non-alkoxylated) alcohol, diol and/or triol from which the alkoxylate is respectively derived, along with other carbon chain length alcohols, diols and/or triols, typically in a lower concentration than the basic alcohol, diol and/or triol respectively. Although it is stated that the composition “comprises essentially of” the alkanol alkoxylate, diol alkoxylate and/or triol alkoxylate, further additional substances, for example pharmaceutically acceptable excipients, may also be included in the composition but are not relevant for considering the definition of purity considered herein.

Even if the biologically active agent of the composition were to include up to 55% of the basic alcohol, diol and/or triol as discussed above, e.g. lauryl alcohol which has been determined in the prior art to have potent antimicrobial properties, this amount would not lead to the antimicrobial effects that have been observed and which will be described in more detail below.

Advantageously, the biologically active agent of the composition may have a GC purity of at least 50%, preferably of at least 60%, further preferably of at least 70% and yet further preferably of at least 80%. Indeed, the biologically active agent of the composition may have a GC purity of at least 90%, possibly greater than 95%, for example 97%.

Preferably the composition comprises essentially of two or more alkanol alkoxylates, diol alkoxylates and/or triol alkoxylates, i.e. the composition may be a mixture of two or more of said alkoxylates forming the biologically active agent, in which case each alkanol/diol/triol alkoxylate may have a GC purity of at least 45%.

An embodiment of the invention is wherein the composition consists essentially of one or more pure alkanol alkoxylate, diol alkoxylate and/or triol alkoxylate, as defined herein.

The alkanol alkoxylate of the composition may be formed by alkoxylating an alkanol having the molecular formula:

CₙH₂ₙ₊₂O

wherein n≧12. Preferably however n may be in the range of from 12 to 25. Outside of this range, the antimicrobial effects that have been observed (to be described below) seem to decrease to the point where no clinically worthwhile positive effect is exhibited. Similarly, each of the diol alkoxylates and triol alkoxylates may be formed by alkoxylating a corresponding diol or triol respectively.

Preferably the alkanol/diol/triol from which the alkoxylate is derived is unsaturated, possibly multiply-unsaturated; said unsaturation may survive into the final alkoxylate. Advantageously the alkanol may have the molecular formula C₁₂H₂₆O (i.e. n=12; lauryl alcohol), C₁₄H₃₀O (i.e. n=14; myristyl alcohol), C₁₆H₃₄O (i.e. n=16; cetyl alcohol), or C₁₈H₃₈O (i.e. n=16; stearyl alcohol). Advantageously, each of the diol (having two hydroxyl groups) and the triol (having three hydroxyl groups) may have a twelve-carbon chain as their molecular backbones. Furthermore, the alkanol/diol/triol from which the alkoxylate is derived may be substituted (in one or more locations) along the carbon chain with other atoms, e.g. oxygen or nitrogen.

Preferentially the alkanol alkoxylate in the composition of the invention may be formed by ethoxylating the alkanol, i.e. by adding one or more (m) ethoxy-functional groups (C₂H₄O) into the alkanol (CₙH₂ₙ₊₂O) according to the following reaction:

CₙH₂ₙ₊₂O+mC₂H₄O→CₙH₂ₙ₊₁(OC₂H₄)ₘOH

Each of the diol alkoxylates and triol alkoxylates may be formed by ethoxylation of the corresponding diol and triol respectively in the same manner.

Typically between 1 and 15 ethoxy-functional groups may be present in the ethoxylated alkanol/diol/triol, i.e. 1<m<15. However preferably, between 1 and 8 ethoxy-functional groups may be present in the ethoxylated alkanol, i.e. 1<m<8, and further preferably either 4, 5 or 6 ethoxy-functional groups may be present in the ethoxylated alkanol, i.e. m=4, m=5 or m=6. Preferably, between 1 and 10 ethoxy-functional groups may be present in the ethoxylated diol and/or triol, i.e. 1<m<10, with the ethoxy-groups being located at either one or both ends of the carbon chain. A narrow-range alkoxylation catalyst, e.g. zirconium dodecanoxide sulphate, may be used to narrow the distribution of the alkoxylated product, i.e. to control m.

Alternatively, the pure alkanol/diol/triol alkoxylates of the invention may be formed by any other suitable route known to the skilled man, whether on an industrial scale or otherwise, which would result in a product of the required purity, including condensation of a polyethylene glycol (PEG) of known chain length with an alkanol, or condensation of block-copolymers (such as PEG and polypropylene glycol (PPG)) with an alkanol.

Regarding the measured antimicrobial effect of the composition of the invention, this may be quantified by its minimum inhibitory concentration (MIC). The MIC of an antimicrobial compound is the lowest concentration of it that will inhibit visible growth of a micro-organism after overnight incubation. The composition of the invention preferably has an MIC of less than approximately 0.88 mmol/L.

However, further preferably the MIC exhibited may be in the range of from approximately 0.055 to approximately 0.44 mmol/L, and most preferably less than approximately 0.22 mmol/L.

The present invention concerns a composition for use in the treatment of microbial infection and also for use in the prevention of microbial infection.

The composition of the invention can generally be used to treat a microbial infection. By this we include that, when administered to a subject in need thereof, the composition alleviates one or more symptoms of the infection, i.e. to cure the subject of the infection, and/or to reduce the effects of the infection on the patient.

By “microbial infection”, we include that the composition of the first aspect of the invention can be used to treat an infection caused by micro-organisms, including fungal, viral and bacterial infections.

Preferably the infection is caused by bacteria and the composition of the invention can be considered an antibacterial composition. By this we include that the composition can kill (bactericide) or inhibit the growth of (a bacteriostatic) bacterial cells. By “subject” we preferably mean a human.

As way of example, the composition can be used in the treatment of an infection caused by any one or more of the following micro-organisms: Escherichia coli, Klebsiella pneumoniae, Providencia rettgeri, Enterobacter cloacae, Serratia marcescens, Salmonella typhimurium, Pseudomonas aeruginosa, Yersinia enterocolitica, Burkholderia cepacia, Acinetobacter baumannii, Steptococcus pyogenes, Staphylococcus aureus (MRSA), Staphylococcus aureus, Staphylococcus epidermidis, Listeria monocytogenes, Enterococcus faecium, Enterococcus faecalis, Bacillus subtilis, Candida albicans, Candida glabrata, Enterococcus faecalis (VRE), Enterococcus faecium (VRE), Enterococcus casseliflavus (VRE), Clostridium difficile. It is particularly preferred that the infection is caused by Methicillin-resistant Staphylococcus aureus (MRSA) and Vancomycin-Resistant Enterococcus (VRE).

Therefore as can be appreciated the composition has much utility for the treatment of a wide range of different bacterial infections. The infections to be treated include MRSA and VRE mediated infection.

Examples of microbial infections which can be treated with the composition of the invention, along with preferred means of administration are provided below:

S. aureus—Atopic eczema, scalded skin syndrome, boils and abscesses (topical administration); Toxic shock syndrome (systemic administration).

S. epidermidis infection (topical administration); CSF shunt infection (systemic administration).

S. pyogenes—Necrotizing fasciitis, cellulitis, impetigo (topical administration); streptococcal sore throat, Rheumatic fever, Scarlet fever (systemic administration).

E. coli infection (topical administration); Meningitis/septicaemia (systemic administration).

L. monocytogenes infection (topical administration); Meningitis/septicaemia (systemic administration).

E. faecium infection (topical administration); Neonatal meningitis (systemic administration).

E. faecalis infection (topical administration); Endocarditis; bladder, prostate and epididymal infections (systemic administration).

C. albicans infection (topical administration); fungal infections in immune-compromised individuals (systemic administration).

C. glabrata infection, including infections in immune-compromised individuals (e.g. those with HIV) (topical administration).

Clostridium difficile infection (topical administration). Particularly preferred is where the composition of the first aspect of the invention is incorporated into cleansing preparations, such as syndets (synthetic detergents), soaps and other washing preparations).

A preferred embodiment of the invention is where the infection to be treated is a superinfection. By “superinfection” we include any infection following a previous infection, especially when caused by microorganisms that are resistant or have become resistant to the antibiotics used earlier. Examples of the superinfections which can be treated with the composition of the invention include Methicillin-resistant Staphylococcus aureus (MRSA) and Vancomycin-Resistant Enterococcus (VRE) infections.

As can be seen from the accompanying examples, the inventors have demonstrated that the compositions of the invention are surprisingly effective for treating MRSA and VRE infections. In deriving this data, the inventors deliberately selected the most problematic strains of MRSA and VRE, and nevertheless showed that the compositions of the invention can successfully act to inhibit the growth of these strains.

As mentioned above, the emergence of MRSA and VRE in recent years, and the rise in associated infections, has lead to an urgent search to identify new compositions which can be used to treat infections caused by such bacteria. The compositions of the invention have much utility in for the treatment of such infections. Different routes of administration can be used to administer the composition of the invention for treating MRSA or VRE superinfection, including topical and systemic.

In addition to the treatment of superinfections, the present inventors have demonstrated that the disclosed composition of the invention also has much utility for the treatment of skin infections including impetigo, eczema, and acne.

In addition to the different types of infection which can be treated with the composition of the invention, the inventors have also determined that the composition can be prepared into a range of different formulations appropriate for separate means of administration to a subject. A discussion is provided below as to the different types of formulations which can be prepared.

However, preferably the composition is formulated for systemic or topical administration to subject.

As mentioned above, by “microbial infection”, we include that the composition of the first aspect of the invention can be used to prevent and/or treat a viral infection.

As demonstrated in the accompanying examples, the inventors have shown that a representative alkoxylated alkanol compound (Laureth 4) has antiviral activity: the compound can alleviate the cytopathic effect of virus on cells. By “antiviral activity” we include where the composition of the first aspect of the invention alleviates the cytopathic effect of virus on cells by protecting the cells from the effect of virus infection, i.e. the composition has cellular protective activity. As can be appreciated, by protecting cells from damage caused by viral infection then the composition of the invention can provide a significant benefit to the patient being treated since may aspects of viral infection reflect cell damage caused by the viral infection, e.g. inflammatory responses to respiratory viral infection.

We also include where the composition of the first aspect of the invention destroys the viral particles, i.e. the composition is a viricide.

Preferably the viral infection is caused by Respiratory Syncytial Virus (RSV).

The present invention generally relates to the application of the composition of the invention as a medicament. There now follows a discussion as to how the composition can be formulated as a pharmaceutical composition, means of administering the composition, and suggested dosage regimes. Any reference below to an ‘agent’ should be interpreted as referring to the biological active agent within the antimicrobial composition of the invention.

It will be appreciated that the amount of a composition needed according to the invention is determined by biological activity and bioavailability which in turn can depend on the mode of administration and the physicochemical properties of the agent. The frequency of administration will also be influenced by the abovementioned factors and particularly the half-life of the agent within the target tissue or subject being treated.

Known procedures, such as those conventionally employed by the pharmaceutical industry (e.g. in vivo experimentation, clinical trials etc), may be used to establish specific formulations of the agents and precise therapeutic regimes (such as daily doses and the frequency of administration).

Generally, a daily dose of between 0.01 g/kg of body weight and 0.1 g/kg of body weight of the composition of the invention may be used in a treatment regimen for systemic administration; more preferably the daily dose is between 0.01 mg/kg of body weight and 100 mg/kg of body weight.

Daily doses may be given as a single administration (e.g. a single daily injection or a single dose from an inhaler). Alternatively the agent (e.g. an antibody or aptamer) may require administration twice or more times during a day.

Medicaments should comprise a therapeutically effective amount of the composition and a pharmaceutically acceptable vehicle. A “therapeutically effective amount” is any amount of an agent which, when administered to a subject leads to an improvement in the microbial infection.

A “subject” may be a vertebrate, mammal, domestic animal or human being. It is preferred that the subject to be treated is human. When this is the case the agents may be designed such that they are most suited for human therapy. However it will also be appreciated that the agents may also be used to treat other animals of veterinary interest (e.g. horses, cattle, dogs or cats).

A “pharmaceutically acceptable vehicle” as referred to herein is any physiological vehicle known to those skilled in the art as useful in formulating pharmaceutical compositions.

In one embodiment, the medicament may comprise between about 0.01 μg and 0.5 g of the agent. More preferably, the amount of the agent in the composition is between 0.01 mg and 200 mg, and more preferably, between approximately 0.1 mg and 100 mg, and even more preferably, between about 1 mg and 10 mg. Most preferably, the composition comprises between approximately 2 mg and 5 mg of the agent.

Preferably, the medicament comprises approximately 0.1% (w/w) to 90% (w/w) of the agent, and more preferably, 1% (w/w) to 10% (w/w). The rest of the composition may comprise the vehicle.

Pharmaceutical compositions can have a number of different forms depending, in particular on the manner in which the composition is to be used. Thus, for example, the composition may be in the form of a powder, tablet, capsule, liquid, ointment, cream, gel, hydrogel, aerosol, emulsion, spray, micelle, transdermal patch, liposome or any other suitable form that may be administered to a person or animal. It will be appreciated that the vehicle of the composition should be one which is well tolerated by the subject to whom it is given, and preferably enables delivery of the therapeutic to the target cell, tissue, or organ. The composition can also be incorporated into cleansing preparations, such as syndets (synthetic detergents), soaps and other washing preparations. Methods of preparing such pharmaceutical compositions are well known in the art and can be readily used by the skilled person to prepare the stated formulations.

In a preferred embodiment, the pharmaceutical vehicle is a liquid and the pharmaceutical composition is in the form of a solution. In another embodiment, the pharmaceutical vehicle is a gel and the composition is in the form of a cream or the like.

By way of a first example, in one embodiment of the invention the composition is wherein Laureth-3 (1.6 g) is dissolved in isopropyl palmitate (98.4 g), and is suitable as a bath additive (in >100 litres of water) to assist in the treatment of Staphylococcus aureus (and other) skin infections, including atopic eczema.

Compositions comprising such therapeutic entities may be used in a number of ways. For instance, systemic administration may be required in which case the entities may be contained within a composition that may, for example, be ingested orally in the form of a tablet, capsule or liquid. Alternatively, the composition may be administered by injection into the blood stream. Injections may be intravenous (bolus or infusion) or subcutaneous (bolus or infusion). The entities may be administered by inhalation (e.g. intranasally).

Therapeutic entities may also be incorporated within a slow or delayed release device. Such devices may, for example, be inserted on or under the skin, and the compound may be released over weeks or even months. Such devices may be particularly advantageous when long term treatment with an entity is required and which would normally require frequent administration (e.g. at least daily injection).

A preferred embodiment of the invention is wherein the composition of the invention further comprises one or more further antimicrobial agents. Alternatively, the composition of the invention is packaged and presented in association with, one or more further antibiotics.

As can be seen in the accompanying examples, the inventors have determined that the composition of the invention can provide a synergistic enhancement of the antimicrobial function of presently known antibiotics. This is wholly unexpected from the art, and could not have been appreciated or anticipated from the existing knowledge of the components of the composition of the invention or from the activity or behaviour of known antibiotics.

A preferred embodiment is wherein the antimicrobial agent is Vancomycin, Rifampicin, Trimethoprim, Moxyfloxacin, Fusidic Acid, Muprocin, Clindamycin, Cefoxitin, Gentamicin, Chloramphenicol, Tetracycline or Erythromycin. Most preferably the antibiotic is Cefoxitin, and the composition of the invention is a Laureth derivative.

The amount of the antibiotic used in combination with the compositions of the invention will vary depending on the specific antibiotic and composition, the infection to be treated, and the mode of administration, as can be appreciated by the skilled person.

As can be appreciated, not only can the composition of the invention be formulated to include the additional antibiotic as mentioned above, the composition can be prepared for administering to a subject that has previously separately been administered the antibiotic, or that will receive the antibiotic. All such potential combinations of the composition of the invention with the previously known antibiotic are contemplated and intended to be encompassed by the term “packaged and presented in association with, one or more further antibiotics”.

A further aspect of the invention provides a pharmaceutical composition comprising the composition as claimed in any of the previous claims and a pharmaceutically acceptable excipient.

Examples of pharmaceutically acceptable excipient (or vehicles) are discussed above.

A further aspect of the invention provides the use of a composition as defined above for the manufacture of a medicament for treating a microbial infection.

A further aspect of the invention provides a method of treating a microbial infection comprising administering to a subject in need thereof a composition as defined above.

A preferred embodiment of these aspects of the invention is wherein medicament is used in association with one or more of the further antimicrobial agents as defined above in relation to the first aspect of the invention, or the subject has been, is, or will be administered with one or more said antimicrobial agents.

A further aspect of the invention provides a composition for use in treating microbial infection or a method or use substantially as hereinbefore described.

All of the features described herein (including any accompanying claims, abstract and drawings), and/or all of the steps of any method or process so disclosed, may be combined with any of the above aspects in any combination, except combinations where at least some of such features and/or steps are mutually exclusive.

### EXAMPLES AND DATA

For a better understanding, the present invention will now be more particularly described, by way of non-limiting example only, as follows. The micro-organisms shown in Table I overleaf were acquired from the National Collection of Type Cultures (NCTC), Colindale, United Kingdom; the American Type Culture Collection (ATCC), Manassas, United States and the National Collection of Pathogenic Fungi (NCPF), Colindale, United Kingdom. They include Gram negative bacteria, Gram positive bacteria and pathogenic yeasts.

To determine the MIC of a range of compositions according to the invention in respect of each of the micro-organisms labelled as shown in Table I below, the following experiment was conducted. Throughout the period of testing the microbial strains were subcultured and incubated for 18-20 hours at 37° C. on a weekly basis. Horse blood agar plates were used to sustain the micro-organisms both prior to and throughout testing. The alkoxylated alkanols listed in Table II below were obtained (these are obtainable from Sigma-Aldrich Company Ltd, Dorset, England), each having a GC purity of at least 97%. In addition, a series of the alkanols (un-ethoxylated) were also obtained; these are listed in Table III below.

TABLE I

| Micro-organism | NCTC/ATCC/NCPF | |
|---|---|---|
| Number | Reference | Micro-organism Name |
| 1 | 10418 | Escherichia coli |
| 3 | 7475 | Providencia rettgeri |
| 11 | 8306 | Steptococcus pyogenes |
| 12 | 11939 | Staphylococcus aureus (MRSA) |
| 13 | 6571 | Staphylococcus aureus |
| 14 | 11047 | Staphylococcus epidermidis |
| 15 | 11994 | Listeria monocytogenes |
| 16 | 7171 | Enterococcus faecium |
| 17 | 775 | Enterococcus faecalis |
| 18 | 9372 | Bacillus subtilis |
| 19 | 90028 | Candida albicans |
| 20 | 3943 | Candida glabrata |

TABLE II

| Alkoxylated | Relative | |
|---|---|---|
| Alkanol | Molecular | |
| Reference | Mass (Mᵣ) | Alkoxylated Alkanol Name |
| D1 | 202.34 | Ethylene Glycol Monodecyl Ether (Deceth-1) [C₁₀E₁] |
| L1 | 230.11 | Ethylene Glycol Monododecyl Ether (Laureth-1) [C₁₂E₁] |
| L2 | 274.44 | Diethylene Glycol Monododecyl Ether (Laureth-2) [C₁₂E₂] |
| L3 | 318.49 | Triethylene Glycol Monododecyl Ether (Laureth-3) [C₁₂E₃] |
| L4 | 362.55 | Tetraethylene Glycol Monododecyl Ether (Laureth-4) [C₁₂E₄] |
| L5 | 406.61 | Pentaethylene Glycol Monododecyl Ether (Laureth-5) [C₁₂E₅] |
| L6 | 450.66 | Hexaethylene Glycol Monododecyl Ether (Laureth-6) [C₁₂E₆] |
| L7 | 494.72 | Heptaethylene Glycol Monododecyl Ether (Laureth-7) [C₁₂E₇] |
| L8 | 538.77 | Octaethylene Glycol Monododecyl Ether (Laureth-8) [C₁₂E₈] |
| M4 | 390.61 | Tetraethylene Glycol Monotetradecyl Ether (Myristeth-4) [C₁₄E₄] |
| M8 | 566.82 | Octaethylene Glycol Monotetradecyl Ether (Myristeth-8) [C₁₄E₈] |

TABLE III

| Prior Art | Relative | |
|---|---|---|
| Alkanol | Molecular | |
| Reference | Mass (Mᵣ) | Alkanol Name |
| D0 | 158.28 | Decyl Alcohol (1-Decanol) [C₁₀] |
| L0 | 186.33 | Lauryl Alcohol (1-Dodecanol) [C₁₂] |
| M0 | 214.39 | Myristyl Alcohol (1-Tetradecanol) [C₁₄] |
| C0 | 242.44 | Cetyl Alcohol (1-Hexadecanol) [C₁₆] |
| S0 | 270.49 | Stearyl Alcohol (1-Octadecanol) [C₁₈] |

For the avoidance of doubt, the nomenclature shown in the square brackets in Tables II and III above, Tables IV, VII and VIII below, and throughout the remainder of this specification, is another way of easily representing the entity in question by reference to the number of carbon atoms (C) in the chain and the number of ethoxy-groups (E) attached thereto (if any).

The experimental procedure for MIC testing was as follows.

n-Alkanol and Ethoxylated Derivative Preparation

Molar equivalent concentrations were used to allow comparison between compounds of differing molecular mass. Due to weighing difficulties, any compound in solid form was melted in a water bath prior to dilution. To make ten-times strength concentrations, which takes into account agar dilution, 0.0352 mmol of each compound was weighed into plastic universal bottles and 4 mL of sterile distilled water (SDW) was added. To produce a homogenous emulsion the universals were vortexed; once achieved the emulsions were serial diluted by adding 2 mL to 2 mL of SDW.

Myristyl alcohol and lauryl alcohol were re-weighed and dissolved in 25 μL of the polar solvent 1-methyl-2-pyrrolidone (M6762; Sigma-Aldrich) in sterile glass universals due to an inability to achieve an even suspension. To maintain an even emulsion in the presence of SDW 37.5 μL of the non-ionic detergent Tween 20 (P1379, Sigma-Aldrich) was also added. 3.9375 mL of SDW was added to these, to create a 4 mL volume; they were vortexed and diluted as before.

### Agar Preparation

For every 100 mL of Isosensitest agar (IST) (CM0471; Oxoid, Basingstoke, England), 3.1 g of the IST powder was added to 100 mL SDW (as per manufacturer's instructions) and sterilised for 15 minutes at 121° C. Once sterilised, the molten agar was cooled to 50° C. in a water-bath. Subsequently 4.8 g of CCEY powder (BC2160; Bioconnections, Wetherby, England), also known as Brazier's medium, was added to 100 mL SDW for every 100 mL of agar needed. This was then sterilised for 15 minutes at 121° C. Once sterilised the molten agar was cooled to 50° C. in a water-bath and 4 mL of egg-yolk emulsion (S2073; TCS Biosciences Ltd, Buckingham, United Kingdom) and 1 mL of lysed defibrinated horse blood (HB035; TCS Biosciences Ltd, Buckingham, United Kingdom) were also added for every 100 mL needed. Horse blood lysis is achieved by dilution to half concentration with SDW; when fully lysed the mixture will turn dark red in colour.

18 mL agar was added to each universal and then poured into sterile Petri dishes. This step creates a 1/10 dilution resulting in agar plates of the required strength. Control plates for each set of strains were created by adding 18 mL of agar to 2 mL of SDW.

### Preparation of Inocula for MIC

Suspensions of each bacterial species were prepared with a density equal to that of a 0.5 McFarland standard using a densitometer. All suspensions were made using sterile distilled water and fresh 18-20 hour cultures. As a 0.5 McFarland standard contains 1.5×10⁸ CFU/mL and the multipoint inoculators applies 1 μL spots of liquid, each bacterial suspension was diluted 1/15 (20 μL suspension to 280 μL sterile distilled water).

### Inoculation of Agar

Bacterial suspensions were prepared from fresh 18-24 hour cultures as previously described. 1 μL of each strain was inoculated onto all 100 agar plates containing a different chemical concentration and the 2 control plates. This was done using a multipoint inoculator which inoculates 20 strains per plate. All plates were incubated for 22 hours at 37° C. in aerobic conditions.

### Results

Each plate was examined for bacterial growth after 22 and 48 hours, with each being incubated in between examinations. A positive result was recorded as growth of ≧10 colonies. A negative result was recorded as growth of <10 colonies as this is equivalent to 99.9% inhibition. The lowest concentration of chemical that inhibited growth was recorded as the MIC. The results are recorded in Table IV below, where the numbers are quoted in mmol/L.

TABLE IV

| Micro- | | | | | | | | | | | | |
|---|---|---|---|---|---|---|---|---|---|---|---|---|
| organism | D0 [C₁₀] | | D1 [C₁₀E₁] | | L0 [C₁₂] | | L1 [C₁₂E₁] | | L2 [C₁₂E₂] | | L3 [C₁₂E₃] | |
| Number | 22 hrs | 48 hrs | 22 hrs | 48 hrs | 22 hrs | 48 hrs | 22 hrs | 48 hrs | 22 hrs | 48 hrs | 22 hrs | 48 hrs |
| 1 | >0.88 | >0.88 | >0.88 | >0.88 | >0.88 | >0.88 | 0.22 | 0.22 | >0.88 | >0.88 | >0.88 | >0.88 |
| 3 | >0.88 | >0.88 | >0.88 | >0.88 | >0.88 | >0.88 | >0.88 | >0.88 | >0.88 | >0.88 | >0.88 | >0.88 |
| 11 | >0.88 | >0.88 | >0.88 | >0.88 | 0.44 | >0.88 | 0.22 | 0.22 | 0.11 | 0.11 | ≦0.05 | ≦0.05 |
| 12 | >0.88 | >0.88 | >0.88 | >0.88 | 0.44 | >0.88 | 0.44 | 0.44 | 0.11 | 0.11 | 0.11 | 0.11 |
| 13 | >0.88 | >0.88 | >0.88 | >0.88 | 0.44 | 0.44 | 0.22 | 0.22 | 0.11 | 0.11 | ≦0.05 | ≦0.05 |
| 14 | >0.88 | >0.88 | >0.88 | >0.88 | >0.88 | >0.88 | 0.22 | 0.22 | 0.11 | 0.11 | ≦0.05 | ≦0.05 |
| 15 | >0.88 | >0.88 | >0.88 | >0.88 | >0.88 | >0.88 | 0.44 | 0.88 | 0.22 | 0.22 | 0.11 | 0.22 |
| 16 | >0.88 | >0.88 | >0.88 | >0.88 | >0.88 | >0.88 | >0.88 | >0.88 | >0.88 | >0.88 | 0.11 | 0.11 |
| 17 | >0.88 | >0.88 | >0.88 | >0.88 | >0.88 | >0.88 | 0.44 | 0.44 | 0.22 | 0.22 | 0.11 | 0.11 |
| 18 | >0.88 | >0.88 | >0.88 | >0.88 | >0.88 | >0.88 | 0.44 | 0.44 | 0.11 | 0.22 | 0.11 | 0.11 |
| 19 | >0.88 | >0.88 | >0.88 | >0.88 | >0.88 | >0.88 | 0.44 | 0.44 | 0.22 | 0.22 | 0.22 | 0.22 |
| 20 | >0.88 | >0.88 | >0.88 | >0.88 | 0.44 | >0.88 | 0.22 | 0.22 | 0.11 | 0.11 | 0.11 | 0.11 |
| Micro- | | | | | | | | | | | | |
| organism | L4 [C₁₂E₄] | | L5 [C₁₂E₅] | | L6 [C₁₂E₆] | | L7 [C₁₂E₇] | | L8 [C₁₂E₈] | | M0 [C₁₄] | |
| Number | 22 hrs | 48 hrs | 22 hrs | 48 hrs | 22 hrs | 48 hrs | 22 hrs | 48 hrs | 22 hrs | 48 hrs | 22 hrs | 48 hrs |
| 1 | >0.88 | >0.88 | >0.88 | >0.88 | >0.88 | >0.88 | >0.88 | >0.88 | >0.88 | >0.88 | >0.88 | >0.88 |
| 3 | >0.88 | >0.88 | >0.88 | >0.88 | >0.88 | >0.88 | >0.88 | >0.88 | >0.88 | >0.88 | >0.88 | >0.88 |
| 11 | ≦0.05 | ≦0.05 | ≦0.05 | ≦0.05 | ≦0.05 | ≦0.05 | 0.11 | 0.11 | ≦0.05 | ≦0.05 | 0.22 | 0.22 |
| 12 | 0.11 | 0.11 | 0.11 | 0.22 | 0.11 | 0.11 | 0.22 | 0.22 | 0.22 | 0.44 | 0.11 | 0.11 |
| 13 | 0.11 | 0.11 | 0.11 | 0.11 | 0.11 | 0.11 | 0.11 | 0.11 | 0.11 | 0.11 | 0.44 | 0.44 |
| 14 | 0.11 | 0.11 | 0.11 | 0.11 | 0.11 | 0.11 | 0.11 | 0.11 | 0.11 | 0.11 | 0.11 | 0.22 |
| 15 | 0.11 | 0.22 | 0.11 | 0.22 | 0.11 | 0.11 | 0.22 | 0.22 | 0.44 | >0.88 | >0.88 | >0.88 |
| 16 | 0.22 | 0.22 | 0.22 | 0.22 | 0.44 | 0.22 | >0.88 | >0.88 | >0.88 | >0.88 | >0.88 | 0.22 |
| 17 | 0.11 | 0.11 | 0.11 | 0.11 | 0.11 | 0.11 | 0.11 | 0.11 | 0.11 | 0.11 | >0.88 | 0.11 |
| 18 | 0.11 | 0.11 | 0.11 | 0.11 | 0.11 | 0.11 | 0.11 | 0.11 | 0.11 | 0.11 | >0.88 | >0.88 |
| 19 | 0.44 | 0.22 | 0.44 | 0.44 | >0.88 | 0.88 | >0.88 | >0.88 | >0.88 | >0.88 | >0.88 | >0.88 |
| 20 | 0.11 | 0.11 | 0.11 | 0.22 | 0.22 | 0.22 | >0.88 | >0.88 | >0.88 | >0.88 | >0.88 | >0.88 |

| Micro- | | | | | | | | | | |
|---|---|---|---|---|---|---|---|---|---|---|
| organism | M4 [C₁₄E₄] | | M8 [C₁₄E₈] | | C0 [C₁₆] | | C2 [C₁₆E₂] | | S0 [C₁₈] | |
| Number | 22 hrs | 48 hrs | 22 hrs | 48 hrs | 22 hrs | 48 hrs | 22 hrs | 48 hrs | 22 hrs | 48 hrs |
| 1 | >0.88 | >0.88 | >0.88 | >0.88 | >0.88 | >0.88 | >0.88 | >0.88 | >0.88 | >0.88 |
| 3 | >0.88 | >0.88 | >0.88 | >0.88 | 0.11 | 0.11 | >0.88 | >0.88 | >0.88 | >0.88 |
| 11 | 0.0275 | 0.0275 | 0.55 | 0.055 | 0.22 | 0.44 | 0.88 | >0.88 | 0.88 | 0.88 |
| 12 | 0.11 | 0.11 | 0.44 | 0.44 | >0.88 | >0.88 | >0.88 | >0.88 | >0.88 | >0.88 |
| 13 | 0.055 | 0.055 | 0.11 | 0.11 | >0.88 | >0.88 | >0.88 | >0.88 | >0.88 | >0.88 |
| 14 | 0.055 | 0.11 | 0.22 | 0.22 | >0.88 | >0.88 | >0.88 | >0.88 | >0.88 | >0.88 |
| 15 | 0.44 | >0.88 | >0.88 | >0.88 | >0.88 | >0.88 | >0.88 | >0.88 | >0.88 | >0.88 |
| 16 | >0.88 | >0.88 | >0.88 | >0.88 | >0.88 | >0.88 | >0.88 | >0.88 | >0.88 | >0.88 |
| 17 | 0.22 | 0.22 | 0.055 | 0.055 | >0.88 | >0.88 | >0.88 | >0.88 | >0.88 | >0.88 |
| 18 | 0.22 | 0.22 | 0.11 | 0.11 | >0.88 | >0.88 | >0.88 | >0.88 | >0.88 | >0.88 |
| 19 | >0.88 | >0.88 | >0.88 | >0.88 | >0.88 | >0.88 | >0.88 | >0.88 | >0.88 | >0.88 |
| 20 | >0.88 | >0.88 | >0.88 | >0.88 | 0.88 | >0.88 | 0.88 | >0.88 | 0.88 | 0.88 |

On analysis of the results, it becomes clear that no difference was observed in the MIC recorded between decyl alcohol (D0) [C₁₀] and deceth-1 (D1) [C₁₀E₁] in respect of the micro-organisms tested—all results show an MIC of greater than 0.88 mmol/L. Thus decyl alcohol (of molecular formula C₁₀H₂₂O, i.e. n=10) and its alkoxylates derivatives are not within the scope of the present invention.

Turning to the results obtained with lauryl alcohol (L0) [C₁₂] and its ethoxylated derivatives [C₁₂E₁ to C₁₂E₈], it is clear that a significant difference can be achieved in the MIC for different micro-organisms. All MIC values that are less than 0.88 mmol/L are highlighted in bold. Italicisation indicates that the MIC value has increased between the two examinations of the plates at 22 and 48 hours respectively.

Firstly, although the MIC for micro-organisms 11, 12, 13 and 20 are reduced to 0.44 mmol/L for Lauryl alcohol (L0) [C₁₂], it is clear that at the 48 hour re-examination, the MIC has increased to 0.88 mmol/L or greater, thus any short term effect is lost.

Turning to Laureth-1 (L1) [C₁₂E₁], this shows an improved MIC over lauryl alcohol (L0) [C₁₂] with the MIC dropping to 0.44 mmol/L or below, and remaining so at the 48 hour re-examination (with the exception of micro-organism 18 which shows an increased MIC after 48 hours). Further successive improvement in the MIC is observed with Laureth-2 (L2)

[C₁₂E₂] and then each of Laureth-3 (L3) [C₁₂E₃], Laureth-4 (L4) [C₁₂E₄], Laureth-5 (L5) [C₁₂E₆] and Laureth-6 (L6) [C₁₂E₆], with a typical value being 0.11 mmol/L. An improved MIC (over that observed with lauryl alcohol [C₁₂]) is also observed for Laureth-x7 (L7) [C₁₂E₇] and Laureth-8 (L8) [C₁₂E₈], however for these latter ethoxylated alcohols, the effect begins to diminish as fewer micro-organisms show a reduced MIC compared to lauryl alcohol [C₁₂].

A similar reduction in the MIC observed is seen between Myristeth-4 (M4) [C₁₄E₄] and myristyl alcohol (M0) [C₁₄]. An MIC of less than 0.22 mmol/L is typical for some micro-organisms, with values less than 0.11 mmol/L also being observed. An improved MIC (over that observed with myristyl alcohol [C₁₄]) is also observed for Myristeth-8 (M8) [C₁₄E₈], however the effect is not as pronounced as that seen with Myristeth-4 (M4) [C₁₄E₄].

Given the positive performance of the ethoxylates of lauryl alcohol in reducing the MIC for a number of the micro-organisms tested, further MIC testing of Laureth-1 [C₁₂E₁] through to Laureth-8 [C₁₂E₈] was conducted on each of the following:
10 strains of vancomycin-resistant enterococci (VRE)—listed in Table V below; and
20 strains of methicillin-resistant Staphylococcus aureus (MRSA)—listed in Table VI.

TABLE V

| Micro- | | Micro- |
|---|---|---|
| organism | | organism |
| Number | Strain Reference | Name |
| 21 | PHLS URINE 2000 | Enterococcus faecalis (VRE) |
| 22 | 255693 N. Tyneside | Enterococcus faecium (VRE) |
| 23 | Clin waste | Enterococcus casseliflavus (VRE) |
| 24 | 12185 Clin waste | Enterococcus faecium (VRE) |
| 25 | MB 305 | Enterococcus faecium (VRE) |
| 26 | MB 3137 | Enterococcus faecium (VRE) |
| 27 | MB 1969 | Enterococcus faecalis (VRE) |
| 28 | MB 726 | Enterococcus faecalis (VRE) |
| 29 | MB310 | Enterococcus faecium (VRE) |
| 30 | NCTC 12952 | Enterococcus faecium (VRE) |

TABLE VI

| Micro- | | Micro- |
|---|---|---|
| organism | | organism |
| Number | Strain Reference | Name |
| 31 | FIN 54518 (E7) | MRS A |
| 32 | FIN 54511 (E6) | MRS A |
| 33 | BEL 97597 | MRSA |
| 34 | BEL 97598 | MRSA |
| 35 | GER 131/98 (Sger Iidc) | MRSA |
| 36 | GER 1966/97 (Hanover IIIc) | MRSA |
| 37 | FRA 95035 | MRSA |
| 38 | FRA 920 | MRSA |
| 39 | EMRSA 15 19972/98 | MRSA |
| 40 | EMRSA 15 21268/98 | MRSA |
| 41 | EMRSA 15 21698/98 | MRSA |
| 42 | EMRSA 15 1729/98 | MRSA |
| 43 | EMRSA 15 1758/98 | MRSA |
| 44 | EMRSA 16 21354/95 | MRSA |
| 45 | EMRSA 16 03732/95 | MRSA |
| 46 | EMRSA 16 00036/95 | MRSA |
| 47 | EMRSA 16 00998/95 | MRSA |
| 48 | EMRSA 16 18200/95 | MRSA |
| 49 | EMRSA 16 07924/95 | MRSA |
| 50 | NCTC 11939 | MRSA |

The alkanol, alkanol derivatives, agar plates, and inocula were all prepared in the same way as described above.

The plates were examined in the same manner as described above and the results are recorded in Table VII below, where the numbers are quoted in mmol/L.

TABLE VII

| Micro- | | | | | | | | |
|---|---|---|---|---|---|---|---|---|
| organism | L1 [C₁₂E₁] | | L2 [C₁₂E₂] | | L3 [C₁₂E₃] | | L4 [C₁₂E₄] | |
| Number | 22 hrs | 48 hrs | 22 hrs | 48 hrs | 22 hrs | 48 hrs | 22 hrs | 48 hrs |
| 21 | 0.22 | 0.22 | 0.22 | 0.22 | 0.22 | 0.22 | 0.11 | 0.11 |
| 22 | 0.44 | 0.44 | 0.44 | 0.44 | 0.88 | >0.88 | 0.44 | 0.88 |
| 23 | 0.22 | 0.22 | 0.22 | 0.22 | 0.22 | 0.22 | 0.11 | 0.11 |
| 24 | 0.88 | 0.88 | >0.88 | >0.88 | 0.88 | >0.88 | 0.22 | 0.44 |
| 25 | 0.44 | 0.88 | 0.88 | 0.88 | 0.44 | 0.44 | 0.11 | 0.11 |
| 26 | 0.44 | 0.44 | >0.88 | >0.88 | 0.88 | >0.88 | 0.88 | >0.88 |
| 27 | 0.22 | 0.22 | 0.22 | 0.22 | 0.44 | 0.44 | 0.22 | 0.22 |
| 28 | 0.44 | 0.44 | 0.22 | 0.22 | 0.22 | 0.22 | 0.22 | 0.22 |
| 29 | 0.44 | 0.44 | 0.88 | 0.88 | 0.44 | 0.44 | 0.11 | 0.11 |
| 30 | 0.22 | 0.22 | 0.44 | 0.44 | 0.22 | 0.22 | 0.44 | 0.44 |
| 31 | 0.22 | 0.44 | 0.22 | 0.22 | 0.22 | 0.44 | 0.22 | 0.44 |
| 32 | 0.22 | 0.22 | 0.11 | 0.11 | 0.22 | 0.22 | 0.11 | 0.11 |
| 33 | 0.22 | 0.22 | 0.11 | 0.11 | 0.055 | 0.055 | 0.055 | 0.055 |
| 34 | 0.22 | 0.22 | 0.11 | 0.22 | 0.11 | 0.11 | 0.11 | 0.11 |
| 35 | 0.22 | 0.22 | 0.22 | 0.22 | 0.11 | 0.11 | 0.11 | 0.11 |
| 36 | 0.22 | 0.22 | 0.11 | 0.11 | 0.055 | 0.055 | 0.055 | 0.055 |
| 37 | 0.22 | 0.22 | 0.22 | 0.22 | 0.055 | 0.055 | 0.055 | 0.055 |
| 38 | 0.22 | 0.22 | 0.11 | 0.11 | 0.055 | 0.055 | 0.055 | 0.055 |
| 39 | 0.22 | 0.22 | 0.11 | 0.11 | 0.11 | 0.11 | 0.055 | 0.055 |
| 40 | 0.22 | 0.22 | 0.11 | 0.11 | 0.11 | 0.11 | 0.055 | 0.055 |
| 41 | 0.22 | 0.22 | 0.11 | 0.11 | 0.11 | 0.11 | 0.055 | 0.055 |
| 42 | 0.22 | 0.22 | 0.11 | 0.11 | 0.11 | 0.11 | 0.055 | 0.055 |
| 43 | 0.22 | 0.22 | 0.11 | 0.11 | 0.055 | 0.11 | 0.055 | 0.055 |
| 44 | 0.22 | 0.22 | 0.11 | 0.11 | 0.11 | 0.11 | 0.055 | 0.055 |
| 45 | 0.22 | 0.22 | 0.11 | 0.11 | 0.11 | 0.11 | 0.11 | 0.11 |
| 46 | 0.22 | 0.22 | 0.11 | 0.11 | 0.055 | 0.055 | 0.055 | 0.055 |
| 47 | 0.22 | 0.22 | 0.11 | 0.11 | 0.055 | 0.055 | 0.055 | 0.055 |
| 48 | 0.22 | 0.22 | 0.11 | 0.11 | 0.11 | 0.11 | 0.055 | 0.055 |
| 49 | 0.22 | 0.22 | 0.11 | 0.11 | 0.055 | 0.055 | 0.055 | 0.055 |
| 50 | 0.22 | 0.22 | 0.055 | 0.11 | 0.055 | 0.055 | 0.055 | 0.055 |
| Micro- | | | | | | | | |
| organism | L5 [C₁₂E₅] | | L6 [C₁₂E₆] | | L7 [C₁₂E₇] | | L8 [C₁₂E₈] | |
| Number | 22 hrs | 48 hrs | 22 hrs | 48 hrs | 22 hrs | 48 hrs | 22 hrs | 48 hrs |
| 21 | 0.11 | 0.11 | 0.22 | 0.22 | 0.44 | 0.88 | 0.22 | 0.22 |
| 22 | 0.22 | 0.22 | 0.44 | >0.88 | >0.88 | >0.88 | >0.88 | >0.88 |
| 23 | 0.11 | 0.11 | 0.22 | 0.22 | 0.22 | 0.22 | 0.44 | 0.44 |
| 24 | 0.22 | 0.22 | 0.88 | 0.88 | >0.88 | >0.88 | >0.88 | >0.88 |
| 25 | 0.11 | 0.11 | 0.22 | 0.22 | 0.22 | 0.22 | >0.88 | >0.88 |
| 26 | 0.44 | >0.88 | 0.88 | >0.88 | >0.88 | >0.88 | >0.88 | >0.88 |
| 27 | 0.22 | 0.22 | >0.88 | >0.88 | >0.88 | >0.88 | >0.88 | >0.88 |
| 28 | 0.22 | 0.22 | >0.88 | >0.88 | >0.88 | >0.88 | 0.88 | >0.88 |
| 29 | 0.11 | 0.11 | 0.22 | 0.22 | 0.11 | 0.11 | >0.88 | >0.88 |
| 30 | 0.22 | 0.22 | >0.88 | >0.88 | >0.88 | >0.88 | >0.88 | >0.88 |
| 31 | 0.44 | 0.44 | >0.44 | >0.44 | >0.44 | >0.44 | >0.44 | >0.44 |
| 32 | 0.11 | 0.22 | 0.11 | 0.11 | 0.11 | 0.44 | 0.22 | >0.44 |
| 33 | 0.11 | 0.11 | 0.11 | 0.11 | 0.22 | 0.22 | 0.22 | >0.44 |
| 34 | 0.11 | 0.11 | 0.22 | 0.22 | >0.44 | >0.44 | >0.44 | >0.44 |
| 35 | 0.11 | 0.11 | 0.11 | 0.11 | 0.11 | 0.22 | 0.44 | >0.44 |
| 36 | 0.11 | 0.11 | 0.11 | 0.11 | 0.11 | 0.11 | 0.11 | 0.11 |
| 37 | 0.11 | 0.11 | 0.22 | 0.44 | >0.44 | >0.44 | >0.44 | >0.44 |
| 38 | 0.11 | 0.11 | 0.22 | 0.22 | 0.22 | 0.44 | >0.44 | >0.44 |
| 39 | 0.11 | 0.11 | 0.11 | 0.11 | >0.44 | >0.44 | >0.44 | >0.44 |
| 40 | 0.11 | 0.11 | 0.11 | 0.11 | >0.44 | >0.44 | >0.44 | >0.44 |
| 41 | 0.11 | 0.11 | 0.11 | 0.11 | 0.44 | >0.44 | >0.44 | >0.44 |
| 42 | 0.11 | 0.11 | 0.11 | 0.11 | 0.44 | 0.44 | >0.44 | >0.44 |
| 43 | 0.11 | 0.11 | 0.11 | 0.11 | 0.22 | 0.22 | >0.44 | >0.44 |
| 44 | 0.11 | 0.11 | 0.22 | 0.44 | >0.44 | >0.44 | >0.44 | >0.44 |
| 45 | 0.11 | 0.11 | 0.22 | 0.22 | >0.44 | >0.44 | >0.44 | >0.44 |
| 46 | 0.11 | 0.11 | 0.22 | 0.44 | 0.44 | >0.44 | >0.44 | >0.44 |
| 47 | 0.11 | 0.11 | 0.22 | 0.44 | >0.44 | >0.44 | >0.44 | >0.44 |
| 48 | 0.11 | 0.11 | 0.11 | 0.22 | 0.22 | >0.44 | >0.44 | >0.44 |
| 49 | 0.11 | 0.11 | 0.11 | 0.11 | >0.44 | >0.44 | >0.44 | >0.44 |
| 50 | 0.11 | 0.11 | 0.11 | 0.11 | 0.11 | 0.11 | 0.22 | 0.22 |

Turning firstly to the results obtained for the MRSA bacterial samples, it is clear that a significant reduction in MIC can be achieved with each of Laureth-1 [C₁₂E₁] through to Laureth-8 [C₁₂E₈] as compared to the basic lauryl alcohol [C₁₂]. For each sample an MIC of less than 0.44 mmol/L is recorded, often less than 0.22 mmol/L and typically less than 0.11 mmol/L. Laureth-4 [C₁₂E₄] appears to be particularly effective at reducing, and importantly maintaining, the MIC at around 0.055 mmol/L. For the higher molecular weight ethoxylates, especially Laureth-7 [C₁₂E₇] and Laureth-8 [C₁₂E₈] an MIC of 0.44 mmol/L is typically observed; this being maintained between the 22 and 48 hour examination windows.

Moving on to the results obtained for the VRE, it is again clear that a significant difference can be achieved in the MIC for each VRE sample using one or more of the alkanol ethoxylates Laureth-1 [C₁₂E₁] through to Laureth-8 [C₁₂E₈]. Laureth-1 [C₁₂E₁], Laureth-2 [C₁₂E₂], Laureth-3 [C₁₂E₃], Laureth-4 [C₁₂E₄] and Laureth-5 [C₁₂E₅] in particular appear to have a significant effect in reducing the recorded MIC to 0.44 mmol/L or below for most, if not all, of the VRE samples. Furthermore, the reduced MIC appears to remain constant (in most cases) between the 22 and 48 hour examination windows respectively. An improved MIC (over that observed with lauryl alcohol [C₁₂]) is also observed for Laureth-6 [C₁₂E₆] and Laureth-7 [C₁₂E₇] (less so with Laureth-8 [C₁₂E₈]), however for these latter ethoxylated alcohols, the effect begins to diminish as fewer VRE show a reduced MIC compared to lauryl alcohol [C₁₂].

Nonetheless, these results clearly demonstrate the benefit that can be achieved with the present invention in treating infection, such as is caused by the bacteria MRSA, and particularly superinfection, such as is caused by VRE.

### Synergy Testing

Following the experimental evidence determined above, the inventors decided to investigate whether the compositions of the invention had any synergistic effect on the antibacterial action of known antibiotics.

There now follows data relating to a screen for synergistic interaction between Laureth derivatives [C₁₂E₁ to C₁₂E₈] and 12 conventional anti-staphylococcal agents.

Ethoxylated Lauryl Alcohol Derivative Preparation As plates containing quarter strength of the MIC of NCTC 11939 are needed, and therefore very small weights of each compound, they were prepared at twenty time strength in 20 mL volumes in plastic universals. Once prepared these solutions were diluted 1/10 by adding 1 mL to 1 mL SDW, resulting a 2 mL volume at ten times strength. This was undertaken in duplicate to create two identical plates.

### Agar Preparation

IST was prepared as previously stated in above. 18 mL agar was added to each universal and then poured into sterile Petri dishes. Control plates were prepared as stated above

### Preparation of a Lawn of MRSA NCTC 11939

A 0.5 McFarland suspension of MRSA NCTC 11939 was prepared using SDW. This was then adjusted to a concentration of 1.5×107 by adding 200 μL to 1.8 mL of SDW. Once diluted, a sterile cotton swab was used to spread the suspension evenly across the entire surface of each agar plate.

### Application of Antibiotic Impregnated Discs

Twelve antibiotics discs were added to the plates, 6 per plate in a circle. The plates were then incubated aerobically at 37° C.

### Reading the Plates

The diameter of the zones of clearance were measured and recorded in millimetres. The zones for each compound were compared to the control and to BSAC breakpoints.

### Results

The results from the experiment are shown in Table VIII below. A variation of zone size within 2 mm is within acceptable limits of assay reproducibility.

Here it can be seen that the addition of compositions of the invention had a synergistic effect on the effect of antibiotics on the growth of the bacterial strain. In particular, it can be seen that there is enhanced susceptibility to cefoxitin (a surrogate for methicillin) in the presence of all Laureth derivatives. Also L8 [C₁₂E₈] caused a slight increase in susceptibility to various other agents.

Also, importantly no antagonism was observed with any agents.

TABLE VIII
Zone of Inhibition (mm) to various Disc Susceptibility Tests in the presence of various Laureth
derivatives (mmol/L)

| | | L1 | L2 | L3 | L4 | L5 | L6 | L7 | L8 |
|---|---|---|---|---|---|---|---|---|---|
| | Control | [C₁₂E₁] | [C₁₂E₂] | [C₁₂E₃] | [C₁₂E₄] | [C₁₂E₅] | [C₁₂E₆] | [C₁₂E₇] | [C₁₂E₈] |
| Antibiotic | 0 | 0.005 | 0.0275 | 0.01375 | 0.0275 | 0.0275 | 0.0275 | 0.0275 | 0.055 |
| Vancomycin (5 μg) | 20 | 21 | 21 | 19 | 21 | 20 | 20 | 20 | 23 |
| Rifampicin (2 μg) | 36 | 37 | 37 | 37 | 40 | 39 | 40 | 39 | [symbol] |
| Trimethoprim (5 μg) | 27 | 29 | 30 | 28 | 31 | 30 | 31 | 30 | [symbol] |
| Moxyfloxacin (1 μg) | 15 | 14 | 14 | 16 | 14 | 13 | 14 | 18 | [symbol] |
| Fusidic Acid (10 μg) | 35 | 36 | 37 | 35 | 38 | 37 | 39 | 37 | [symbol] |
| Muprocin (5 μg) | 33 | 32 | 33 | 32 | 34 | 32 | 33 | 34 | 35 |
| Clindamycin (2 μg) | 33 | 33 | 32 | 32 | 34 | 33 | 33 | 34 | 35 |
| Cefoxitin (10 μg) | 11 | [symbol] | [symbol] | [symbol] | [symbol] | [symbol] | [symbol] | [symbol] | [symbol] |
| Gentamicin (10 μg) | 28 | 27 | 27 | 27 | 29 | 28 | 30 | 30 | 33 |
| Chloramphenicol (10 μg) | 24 | 22 | 22 | 24 | 23 | 25 | 23 | 22 | 23 |
| Tetracycline (10 μg) | 36 | 34 | 35 | 35 | 36 | 32 | 35 | 36 | [symbol] |
| Erythromycin (5 μg) | 7 | 7 | 7 | 7 | 8 | 7 | 7 | 7 | 8 |

Numbers in Bold and Italics demonstrate a ≧5 mm change

Further experiments have also been conducted to determine the MIC of a number of other compositions as follows. The experimental procedure followed is the same as that initially described at the beginning of this Examples and Data section of the specification, except where indicated below.

Table IX below shows the MIC results obtained with the following:
polyoxyethylene (6) tridecyl alcohol (having n=13; hence C₁₃E₆), which is commercially available as Synperionic™ 13/6;
ceteth-1 (C1) [C₁₆E₁] along with cetyl alcohol (C0) [C₁₆];
15-amino-4-oxa-pentadecanol [AEG1], 3,6,19,22-tetraoxatetracosane-1,24-diol [DEG2], 1-methyl-3-oxapentadecanol [PG1], having the structures shown below, along with 1,12-dodecanediol [D]:

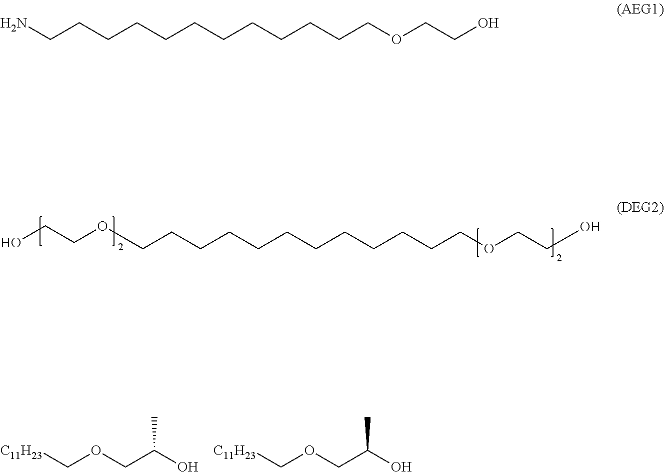

(PG 1)—a racemic mixture of the two enantiomeric forms shown

Although no results for tridecyl alcohol are presented to enable a straight comparison with the C₁₃E₆ ethoxylated version shown in Table IX below, it is clear that in respect of certain of the bacteria tested, polyoxyethylene (6) tridecyl alcohol produces MIC results that are of the same order of magnitude as the results obtained with other alkanol alkoxylates according to the invention, being less than 0.88 mmol/L.

On initial comparison of ceteth-1 [C₁₆E₁] with cetyl alcohol [C₁₆], it would appear that the MIC values are somewhat similar, however it has been observed that in addition to providing MIC results that are the same order of magnitude as cetyl alcohol, ceteth-1 in fact possesses fundamentally different chemical, physical and biological properties as compared to cetyl alcohol, e.g. different HLB, solvent solubilities, emulsifying/solubilising characteristics, percutaneous penetration enhancement, stability, rates of metabolisation, etc. These differences confer properties (and therefore advantages) above and beyond those exhibited by cetyl alcohol, and will likely prove to be beneficial in product formulation.

Similarly on initial comparison of each of AEG1, DEG2 and PG1 with 1,12-dodecanediol, it would again appear that the MIC values are somewhat similar. However it has been observed that in addition to providing MIC results that are the same order of magnitude as 1,12-dodecanediol, each of AEG1, DEG2 and PG1 possesses fundamentally different chemical, physical and biological properties as compared to 1,12-dodecanediol, e.g. different HLB, solvent solubilities, emulsifying/solubilising characteristics, percutaneous penetration enhancement, stability, rates of metabolisation, etc. These differences confer properties (and therefore advantages) above and beyond those exhibited by 1,12-dodecanediol, and will likely prove to be beneficial in product formulation.

TABLE IX

| | NCTC/ | | | | | | | | | | | |
|---|---|---|---|---|---|---|---|---|---|---|---|---|
| Micro- | ATCC / | | | | | | | | | | | |
| organism | NCPF | | C₁₃E₆ | | C₁₆ | | C₁₆E₁ | | D | AEG1 | DEG2 | PG1 |
| Number | Reference | Micro-organism Name | 22 hrs | 48 hrs | 22 hrs | 48 hrs | 22 hrs | 48 hrs | 48 hrs | 48 hrs | 48 hrs | 48 hrs |
| 1 | 10418 | Escherichia coli | >0.88 | >0.88 | >0.88 | >0.88 | >0.88 | >0.88 | >0.88 | 0.88 | >0.88 | >0.88 |
| 2 | 9528 | Klebsiella pneumoniae | >0.88 | >0.88 | 0.88 | 0.88 | >0.88 | >0.88 | >0.88 | >0.88 | 0.88 | >0.88 |
| 3 | 7475 | Providencia rettgeri | >0.88 | >0.88 | 0.11 | 0.11 | >0.88 | >0.88 | >0.88 | 0.88 | >0.88 | 0.88 |
| 4 | 11936 | Enterobacter cloacae | >0.88 | >0.88 | >0.88 | >0.88 | >0.88 | >0.88 | >0.88 | >0.88 | >0.88 | >0.88 |
| 5 | 10211 | Serratia marcescens | >0.88 | >0.88 | >0.88 | >0.88 | >0.88 | >0.88 | >0.88 | >0.88 | >0.88 | >0.88 |
| 6 | 74 | Salmonella typhimurium | >0.88 | >0.88 | 0.88 | 0.88 | >0.88 | >0.88 | >0.88 | >0.88 | >0.88 | >0.88 |
| 7 | 10662 | Pseudomonas aeruginosa | >0.88 | >0.88 | >0.88 | >0.88 | >0.88 | >0.88 | >0.88 | >0.88 | >0.88 | >0.88 |
| 8 | 11176 | Yersinia enterocolitica | >0.88 | >0.88 | >0.88 | >0.88 | >0.88 | >0.88 | >0.88 | >0.88 | >0.88 | >0.88 |
| 9 | 1222 | Burkholderia cepacia | >0.88 | >0.88 | >0.88 | >0.88 | >0.88 | >0.88 | >0.88 | >0.88 | >0.88 | >0.88 |
| 10 | 19606 | Acinetobacter baumannii | >0.88 | >0.88 | >0.88 | >0.88 | >0.88 | >0.88 | >0.88 | >0.88 | >0.88 | >0.88 |
| 11 | 8306 | Steptococcus pyogenes | 0.055 | 0.055 | 0.22 | 0.44 | >0.88 | >0.88 | >0.88 | 0.88 | 0.88 | >0.88 |
| 12 | 11939 | Staphylococcus aureus | 0.11 | 0.11 | >0.88 | >0.88 | >0.88 | >0.88 | >0.88 | >0.88 | >0.88 | 0.88 |
| | | (MRSA) | | | | | | | | | | |
| 13 | 6571 | Staphylococcus aureus | 0.11 | 0.11 | >0.88 | >0.88 | >0.88 | >0.88 | >0.88 | >0.88 | >0.88 | >0.88 |
| 14 | 11047 | Staphylococcus epidermidis | 0.11 | 0.11 | >0.88 | >0.88 | >0.88 | >0.88 | >0.88 | >0.88 | >0.88 | >0.88 |
| 15 | 11994 | Listeria monocytogenes | >0.88 | >0.88 | >0.88 | >0.88 | >0.88 | >0.88 | >0.88 | >0.88 | >0.88 | >0.88 |
| 16 | 7171 | Enterococcus faecium | >0.88 | >0.88 | >0.88 | >0.88 | >0.88 | >0.88 | >0.88 | >0.88 | >0.88 | >0.88 |
| 17 | 775 | Enterococcus faecalis | 0.11 | 0.11 | >0.88 | >0.88 | >0.88 | >0.88 | >0.88 | >0.88 | >0.88 | >0.88 |
| 18 | 9372 | Bacillus subtilis | 0.11 | 0.11 | >0.88 | >0.88 | >0.88 | >0.88 | >0.88 | >0.88 | >0.88 | >0.88 |
| 19 | 90028 | Candida albicans | >0.88 | >0.88 | >0.88 | >0.88 | >0.88 | >0.88 | >0.88 | >0.88 | >0.88 | >0.88 |
| 20 | 3943 | Candida glabrata | >0.88 | >0.88 | 0.88 | >0.88 | 0.88 | 0.88 | >0.88 | >0.88 | >0.88 | >0.88 |

Table X below shows the MIC results obtained with Laureth-4 (C₁₂E₄) with certain Propionibacteria. The results were obtained following incubation for 48 hours at 37° C. in anaerobic conditions.

Again although no results showing the effect of lauryl alcohol (L0) [C₁₂] on the Propionibacteria listed are presented to enable a straight comparison with Laureth-4 (L4), it is clear that in respect of certain of the bacteria tested, Laureth-4 produces MIC results that are of the same order of magnitude as the results obtained when it is applied to certain other microorganisms, being less than 0.88 mmol/L.

TABLE X

| | NCTC/ATCC/NCPF | |
|---|---|---|
| Propionibacterium Name | Reference | L4 [C₁₂E₆] |
| Propionibacterium granulosum | 354039G | 0.44 |
| Propionibacterium granulosum | 354043J | 0.44 |
| Propionibacterium avidum | 340093T | 0.88 |
| Propionibacterium avidum | 340097V | 0.88 |
| Propionibacterium avidum | 354020J | 0.88 |
| Propionibacterium avidum | 340090P | 0.88 |
| Propionibacterium avidum | 354025L | 0.88 |
| Propionibacterium granulosum | 354044V | 0.88 |
| Propionibacterium species | 387327Y | 0.44 |
| Propionibacterium acnes | 737 | 0.88 |

Finally, Table XI below shows the MIC results obtained with Laureth-4 (C₁₂E₄) with certain yeasts. The results were obtained following incubation for 48 hours at 37° C. in aerobic conditions.

Again although no results showing the effect of lauryl alcohol (L0) [C₁₂] on the yeasts listed are presented to enable a straight comparison with Laureth-4 (L4) [C₁₂E₄], it is clear that in respect of all of the yeasts tested, Laureth-4 produces MIC results that are of the same order of magnitude as the results obtained when it is applied to certain other microorganisms, some of which are other yeast variants (as shown in Table IV above), all being less than 0.88 mmol/L.

TABLE XI

| | NCTC/ATCC/NCPF | |
|---|---|---|
| Yeast Name | Reference | L4 [C₁₂E₆] |
| Candida parapsilosis | 381611V | >0.22 |
| Candida parapsilosis | 547133B | >0.22 |
| Candida glabrata | 437713D | >0.22 |
| Candida glabrata | 882870Q | >0.22 |
| Candida lustaniae | 458891B | >0.22 |
| Candida tropicalis | 470273M | >0.22 |
| Candida guilliemondii | 380022E | >0.22 |
| Candida kefyr | 337838T | 0.055 |
| Candida krusei | 378573D | >0.22 |
| Candida dubliniensis | 383086P | 0.11 |
| Candida albicans | 383107 | >0.22 |
| Candida albicans | 384403C | 0.22 |
| Candida albicans | 457932F | >0.22 |
| Candida albicans | 464644V | 0.22 |
| Candida albicans | 561251J | >0.22 |
| Candida albicans | 540668D | 0.11 |
| Candida albicans | 540992V | 0.22 |

### Example 2

### Additional Antibacterial Data

Using the protocols outlined in the example above, the inventors further investigated the antibacterial effects of representative alkoxylated alkanol compounds. The data generated is presented in Table XII below. The inoculum was 10000 cfu/spot; inclubation was for 24 hrs at 37° C.

TABLE XII

| | NCTC/ATCC/ | MIC (%) | |
|---|---|---|---|
| | NCPF | volume/volume | |
| | Reference | L1 | L4 |
| Gram negative bacteria | | | |
| Escherichia coli | 10418 | 0.25 | >4 |
| Klebsiella pneumoniae | 9528 | 0.5 | >4 |
| Acinetobacter baumannii | 19606 | 1 | >4 |
| Stenotrophomonas maltophilia | 10257 | 0.5 | 0.06 |
| Gram positive bacteria | | | |
| (controls) | | | |
| Staphylococcus aureus | 470273M | 0.25 | 0.03 |
| Staphylococcus aureus(MRSA) | 380022E | 0.25 | 0.03 |

The data presented above demonstrates that Stenotrophomonas maltophilia is susceptible to compounds L4 at relatively low concentrations of that agent. At higher concentrations, L1 shows activity against selected Gram-negative species.

### Example 3

### Antiviral Data

The inventors have also investigated the antiviral activity of representative alkoxylated alkanol compounds.

### Protocol

A549 (ATCC CCL-185) cells were plated onto a 96 well and allowed to attach overnight, the wells were washed, and 100 ul of DMEM (Gibco) with 2% FCS was added. To the first column of the data set below, 100 μl of neat virus was added. The virus used was Respiratory Syncytial Virus (RSV), strain A2, a common laboratory strain which retains the ability to infect animal models.

A two serial dilution was performed (up to column 10), lanes 11+12 were uninfected controls, but relevant lanes contained the appropriate concentration of Laureth 4, laureth 4 was added 1 hour after virus absorption. The alkoxylated alkanol compounds used in the experiment were ‘high purity’, i.e. having a GC purity of at least 97%.

After 96 hours incubation at 33° C. the medium was removed, cell washed with PBS and stained with methylene blue (Gibco). Cytopathic effect (cpe) was noted visually and wells marked either as positive or negative when view under a light microscope at 4× magnification.

TABLE XIII

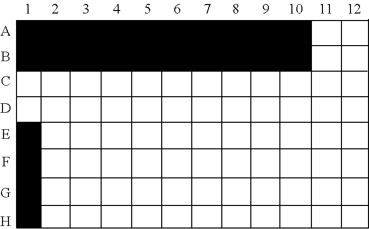

KEY
Columns 1-10 = 2 fold serial dilution of virus prep, starting titre 2 × 10⁶ TCID₅₀
Columns 11 + 12, no virus added, to test effect of Laureth 4 on cells
Rows, A + B No addition of Laureth 4
Rows C + D, Laureth 4 at 1 mM
Rows E + F, Laureth 4 at 0.1 mM
Rows G + H, Laureth 4 at 0.01 mM
Grey cells = cpe evident

The cpe noted in the 0.1 and 0.01, was significantly reduced when compared to the untreated lane. The test was repeated out in three times.

### Comments

It can be seen from the data presented above that Laureth 4 can alleviate the cytopathic effect of the virus on the cells.

It should also be noted that whereas the uninfected cells in the control lane were showing sign of overgrowth the treated, uninfected cells were healthy and showed little cellular apathy suggesting that the Laureth 4 had some benefit upon cellular integrity.

1. A composition for use in the prevention and/or treatment of microbial infection comprising essentially of one or more pure alkanol alkoxylates, diol alkoxylates and/or triol alkoxylates.

2. A composition as claimed in claim 1 wherein the biologically active agent of the composition is a pure alkanol alkoxylate, diol alkoxylate and/or triol alkoxylate, having a GC purity of at least 45%.

3. A composition as claimed in claim 1 comprising essentially of two or more alkanol alkoxylates, diol alkoxylates and/or triol alkoxylates.

4. A composition as claimed in claim 2 wherein each alkanol/diol/triol alkoxylate has a GC purity of at least 45%.

5. A composition as claimed in claim 1 wherein the alkanol alkoxylate is formed by alkoxylating an alkanol having molecular formula:
CₙH₂ₙ₊₂O
wherein n≧12.

6. A composition as claimed in claim 5 wherein n is in the range of from 12 to 25.

7. A composition as claimed in claim 6 wherein n is preferably in the range of from 12 to 18.

8. A composition as claimed in claim 6 wherein n is 12 (lauryl alcohol), 14 (myristyl alcohol), 16 (cetyl alcohol) or 18 (stearyl alcohol).

9. A composition as claimed in claim 1 wherein each of the diol alkoxylate and triol alkoxylate has a twelve-carbon chain backbone.

10. A composition as claimed in claim 1 wherein the alkanol/diol/triol alkoxylate is formed by ethoxylating the respective alkanol/diol/triol.

11. A composition as claimed in claim 10 wherein between 1 and 15 ethoxy-functional groups are present in the ethoxylated alkanol/diol/triol.

12. A composition as claimed in claim 11 wherein between 1 and 8 ethoxy-functional groups are present in the ethoxylated alkanol.

13. A composition as claimed in claim 11 wherein either 4, 5 or 6 ethoxy-functional groups are present in the ethoxylated alkanol.

14. A composition as claimed in claim 13 in the form of Laureth-4. Laureth-5, Laureth-6 or Myristeth-4.

15. A composition as claimed in claim 1 wherein between 1 and 10 ethoxy-functional groups are present in each of the ethoxylated diol and/or ethoxylated triol.

16. A composition as claimed in claim 1 wherein the minimum inhibitory concentration (MIC) exhibited is less than approximately 0.88 mmol/L.

17. A composition as claimed in claim 16 wherein the MIC exhibited is in the range of from approximately 0.055 to approximately 0.44 mmol/L.

18. A composition as claimed in claim 17 wherein the MIC exhibited is less than approximately 0.22 mmol/L.

19. A composition as claimed in claim 1 for use in the treatment of an infection caused by fungal, viral or bacterial micro-organisms.

20. A composition as claimed in claim 1 for use in the treatment of an infection caused by any one or more of the following micro-organisms:
Escherichia coli, Klebsiella pneumoniae, Providencia rettgeri, Enterobacter cloacae, Serratia marcescens, Salmonella typhimurium, Pseudomonas aeruginosa, Yersinia enterocolitica, Burkholderia cepacia, Acinetobacter baumannii, Steptococcus pyogenes, Staphylococcus aureus (MRSA), Staphylococcus aureus, Staphylococcus epidermidis, Listeria monocytogenes, Enterococcus faecium, Enterococcus faecalis, Bacillus subtilis, Enterococcus faecalis (VRE), Enterococcus faecium (VRE), Enterococcus casseliflavus (VRE)Clostridium difficile, Candida albicans, Candida glabrata, Candida parapsilosis, Candida lustaniae, Candida tropicalis, Candida guHliemondii, Candida kefyr, Candida krusei, Candida dubliniensis and certain Propionibacteria; or Respiratory Syncytial Virus (RSV).

21. A composition as claimed in claim 1 for use in the treatment of a superinfection.

22. A composition as claimed in claim 21 wherein the superinfection is caused by Methicillin-resistant Staphylococcus aureus (MRSA) and Vancomycin-Resistant Enterococcus (VRE) infections.

23. A composition as claimed in claim 1 wherein the infection is a skin infection.

24. A composition as claimed in claim 23 wherein the skin infection is including impetigo, eczema or acne.

25. A composition as claimed in claim 1 wherein the composition is formulated for topical or systemic administration.

26. A composition as claimed in claim 1 further comprising one or more further antimicrobial agents.

27. The composition of claim 26 wherein the antimicrobial agent is Vancomycin, Rifampicin, Trimethoprim, oxyfloxacin, Fusidic Acid, Muprocin, Clindamycin, Cefoxitin, Gentamicin, Chloramphenicol, Tetracycline or Erythromycin.

28. A pharmaceutical composition comprising the composition as claimed in claim 1 and a pharmaceutically acceptable excipient.

29. Use of a composition as claimed in claim 1 for the manufacture of a medicament for treating microbial infection.

30. A method of treating a microbial infection comprising administering to a subject in need thereof a composition according to claim 1.

31. The use or method of claim 29 wherein the medicament is used in association with one or more of the antimicrobial agents or the subject has been, is, or will be administered with one or more said antimicrobial agents.

32. (canceled)
